(19) United States
(12) Patent Application Publication
(10) Pub. No.: US 2025/0182325 A1
(43) Pub. Date: Jun. 5, 2025

(54) METHOD AND SYSTEM FOR ESTIMATING A 3D CAMERA POSE BASED ON 2D MASK AND RIDGES AND APPLICATION IN A LAPAROSCOPIC PROCEDURE

(71) Applicant: EDDA TECHNOLOGY, INC., Princeton, NJ (US)

(72) Inventors: Xiaotao Guo, Bronx, NY (US); Guo-Qing Wei, Plainsboro, NJ (US); Li Fan, Belle Mead, NJ (US); Xiaolan Zeng, Princeton, NJ (US); Jianzhong Qian, Princeton Junction, NJ (US)

(21) Appl. No.: 18/528,475

(22) Filed: Dec. 4, 2023

(51) Int. Cl. G06T 7/73; A61B 34/20; G06T 15/20

(52) U.S. Cl. CPC G06T 7/75; A61B 34/20; G06T 15/20; G06T 2207/30096; G06T 2207/30101

(57) ABSTRACT

The present teaching is directed to estimating 3D camera pose based on 2D features detected from a 2D image. Virtual 3D camera poses are generated with respect to a 3D model for a target organ and associated anatomical structures. Virtual 2D images are created by projecting the 3D model from perspectives determined based on the virtual 3D camera poses. Each virtual 2D image includes 2D projected target organ and/or 2D structures of some 3D anatomical structures visible from a corresponding perspective. 2D feature/camera pose mapping models are then accordingly obtained based on 2D features extracted from the virtual 2D images and the corresponding virtual 3D camera poses, where the 2D features include a 2D ridge line projected from a 3D ridge on the target organ represented in the 3D model.

### BACKGROUND

### 1. Technical Field

The present teaching generally relates to computers. More specifically, the present teaching relates to signal processing.

### 2. Technical Background

With the advancement of technologies, more and more tasks are now performed with the assistance of computers. Different industries have benefited from such technological advancement, including the medical industry, where large volume of image data, capturing anatomical information of a patient, may be processed by computers to identify anatomical structures of interest (e.g., organs, bones, blood vessels, or abnormal nodule), obtain measurements for each object of interest (e.g., dimension of a nodule growing in an organ), and quantification of different anatomical structures (e.g., dimension and shape of abnormal nodules). Such information may be used for a wide variety of purposes, including enabling presurgical planning as well as in surgery guidance. Modern laparoscopic procedures may also utilize the technological advancement in the field to obtain information during a surgery to facilitate navigational guide to a surgeon in performing an operation.

This is illustrated in FIG. 1A, where in a setting for laparoscopic procedure, a patient 120 on a surgical table 110 and a laparoscopic camera 130 may be inserted into a patient's body to observe a site of interest to capture, e.g., a surgical instrument 140 (also inserted into the patient's body) appearing at the site, the nearby an organ, and possibly other anatomical structures close to the surgical instrument 140. Two dimensional (2D) images captured by the laparoscopic camera 130 may be displayed (150) so that it may be viewed by a surgeon as a visual guide. The surgeon then mentally corresponds what is seen in 2D images (e.g., a surgical instrument near the surface of an organ) with the actual three-dimensional (3D) object of interest (e.g., the liver to be resected in the operation) to determine which part of the organ is close to the surgical instrument to figure out how to manipulate the surgical instrument.

In a laparoscopic procedure, a 3D model characterizing an organ of interest may be utilized to provide 3D information corresponding to what is seen in 2D images to enhance the effectiveness of visual guide. Such a 3D model may represent both the physical construct of the organ (e.g., a liver) and the anatomical structures inside the organ (e.g., blood vessels, nodule(s) inside a liver). If such a 3D model can be registered with what is seen in 2D images, a projection of such a 3D model at the registered location allows the surgeon to see all 3D objects around or inside the organ. This may provide valuable information to help the surgeon to navigate the surgical tool to achieve the intended task (e.g., remove a nodule) in a manner to avoid causing harm to other parts of the body such as blood vessels.

To utilize a 3D model to introduce enhancement in a laparoscopic procedure, registration of 2D laparoscopic images with a 3D model is needed. In some situations, a surgeon or an assistant may manually select 2D feature points from 2D images and the 3D corresponding points from a 3D model to facilitate registration. However, such a manual approach is impractical in actual surgeries because it is slow, cumbersome, and impossible to do it continuously with changing 2D images while the surgical instrument is moving.

Thus, there is a need for a solution that addresses the challenges discussed above.

### SUMMARY

The teachings disclosed herein relate to methods, systems, and programming for information management. More particularly, the present teaching relates to methods, systems, and programming related to hash table and storage management using the same.

In one example, a method, implemented on a machine having at least one processor, storage, and a communication platform for estimating 3D camera pose based on 2D features detected from a 2D image. Virtual 3D camera poses are generated with respect to a 3D model for a target organ and associated anatomical structures. Virtual 2D images are created by projecting the 3D model from perspectives determined based on the virtual 3D camera poses. Each virtual 2D image includes 2D projected target organ and/or 2D structures of some 3D anatomical structures visible from a corresponding perspective. 2D feature/camera pose mapping models are then accordingly obtained based on 2D features extracted from the virtual 2D images and the corresponding virtual 3D camera poses, where the 2D features include a 2D ridge line projected from a 3D ridge on the target organ represented in the 3D model.

In a different example, a system is disclosed for estimating 3D camera pose based on 2D features detected from a 2D image and includes a camera pose generator and a 2D feature/camera pose mapping model generator. The camera pose generator is provided for generating virtual 3D camera poses with respect to a 3D model previously constructed to model a 3D target organ and 3D anatomical structures associated therewith, wherein each of the virtual 3D camera poses corresponds to a perspective to view the 3D model. The 2D feature/camera pose mapping model generator is provided for creating virtual 2D images corresponding to the virtual 3D camera poses by projecting the 3D model in accordance with corresponding perspectives, wherein each of the virtual 2D images includes 2D projected target organ and/or 2D structures of some of the 3D anatomical structures visible from a corresponding perspective as well as obtaining 2D feature/camera pose mapping models based on the 2D features extracted from the virtual 2D images and the corresponding virtual 3D camera poses. The 2D features include a 2D ridge line projected from a 3D ridge on the target organ represented in the 3D model.

Other concepts relate to software for implementing the present teaching. A software product, in accordance with this concept, includes at least one machine-readable non-transitory medium and information carried by the medium. The information carried by the medium may be executable program code data, parameters in association with the executable program code, and/or information related to a user, a request, content, or other additional information.

Another example is a machine-readable, non-transitory and tangible medium having information recorded thereon for estimating 3D camera pose based on 2D features detected from a 2D image. Virtual 3D camera poses are generated with respect to a 3D model for a target organ and associated anatomical structures. Virtual 2D images are created by projecting the 3D model from perspectives determined based on the virtual 3D camera poses. Each virtual 2D image includes 2D projected target organ and/or 2D structures of some 3D anatomical structures visible from a corresponding perspective. 2D feature/camera pose mapping models are then accordingly obtained based on 2D features extracted from the virtual 2D images and the corresponding virtual 3D camera poses. The 2D features include a 2D ridge line projected from a 3D ridge on the target organ represented in the 3D model.

Additional advantages and novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1A illustrates an exemplary setting for a laparoscopic procedure;

FIG. 1B shows an exemplary 3D model of an organ and a perspective to look into the 3D model from a 3D camera pose;

FIG. 1C shows a 2D image created by projecting a 3D model onto a 2D plane according to a perspective determined by a 3D camera pose;

FIG. 1D illustrates an exemplary 3D model for an organ with a 3D surface and identified ridges, in accordance with an embodiment of the present teaching;

FIGS. 1E-1F illustrates different types of 3D features characterizing an organ, in accordance with an embodiment of the present teaching;

FIG. 1G shows exemplary 2D features that may be observed when a 3D modeled organ is projected to a 2D plane;

FIG. 2 depicts an exemplary high level system diagram of a 3D camera pose estimation framework and utilization thereof, in accordance with an embodiment of the present teaching;

FIG. 3A is a flowchart of an exemplary process to obtain models for mapping 2D features to a 3D camera pose in a 3D camera pose estimation framework, in accordance with an embodiment of the present teaching;

FIG. 3B is a flowchart of an exemplary process to utilize camera pose mapping models to estimate a 3D laparoscopic camera pose based on 2D feature extracted from 2D laparoscopic images, in accordance with an embodiment of the present teaching;

FIG. 4A depicts an exemplary high level system diagram of a 2D feature/camera pose mapping model generator, in accordance with an embodiment of the present teaching;

FIG. 4B illustrates exemplary correspondences between 2D features and 3D camera poses which may be used to build a mapping model, in accordance with an embodiment of the present teaching;

FIG. 4C shows encoded 2D features and 3D camera poses to be used to build a mapping model, in accordance with an embodiment of the present teaching;

FIG. 5A shows an exemplary scheme of encoded 2D features are decoded for reconstructing 2D features for camera pose estimation refinement, in accordance with an embodiment of the present teaching;

FIG. 5B illustrates exemplary types of mapping models that may be trained based on discrete training data, in accordance with an embodiment of the present teaching;

FIG. 5C is a flowchart of an exemplary process for a 2D feature/camera pose mapping model generator, in accordance with an embodiment of the present teaching;

FIG. 6A depicts an exemplary high level system diagram of a camera pose estimator, in accordance with an embodiment of the present teaching;

FIG. 6B is a flowchart of an exemplary process of a camera pose estimator, in accordance with an embodiment of the present teaching;

FIG. 7 is an illustrative diagram of an exemplary mobile device architecture that may be used to realize a specialized system implementing the present teaching in accordance with various embodiments; and

FIG. 8 is an illustrative diagram of an exemplary computing device architecture that may be used to realize a specialized system implementing the present teaching in accordance with various embodiments.

### DETAILED DESCRIPTION

In the following detailed description, numerous specific details are set forth by way of examples in order to facilitate a thorough understanding of the relevant teachings. However, it should be apparent to those skilled in the art that the present teachings may be practiced without such details. In other instances, well known methods, procedures, components, and/or system have been described at a relatively high-level, without detail, in order to avoid unnecessarily obscuring aspects of the present teachings.

The present teaching discloses exemplary methods, systems, and implementations of a framework to estimate a 3D camera pose based on 2D image features detected from a laparoscopic image and an exemplary application in a laparoscopic procedure. A 3D model for an organ may be constructed to represent the organ and various anatomical structures resided therein or nearby in terms of their physical appearances such as dimension, volume, shape, as well as structural features such as ridges thereof. Such a 3D model may be utilized to generate projections with respect to different perspectives of relevant parts in 2D planes. Each of the perspectives may be determined based on corresponding 3D camera poses.

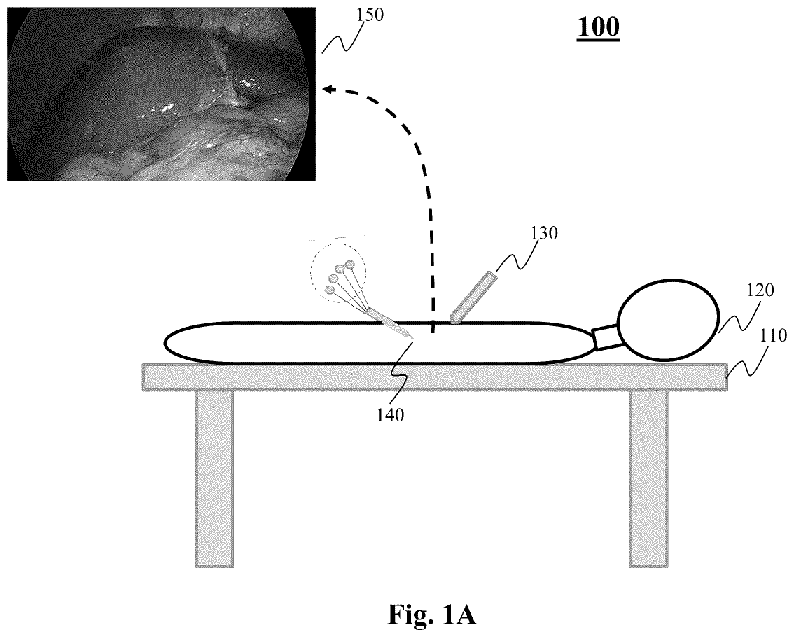

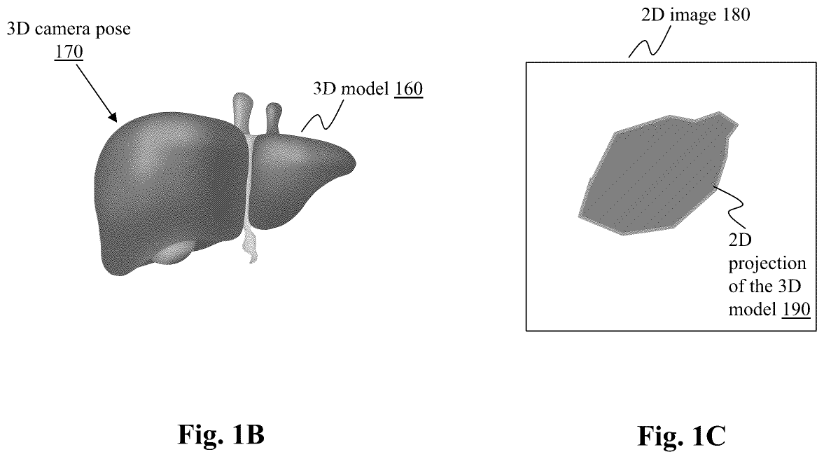

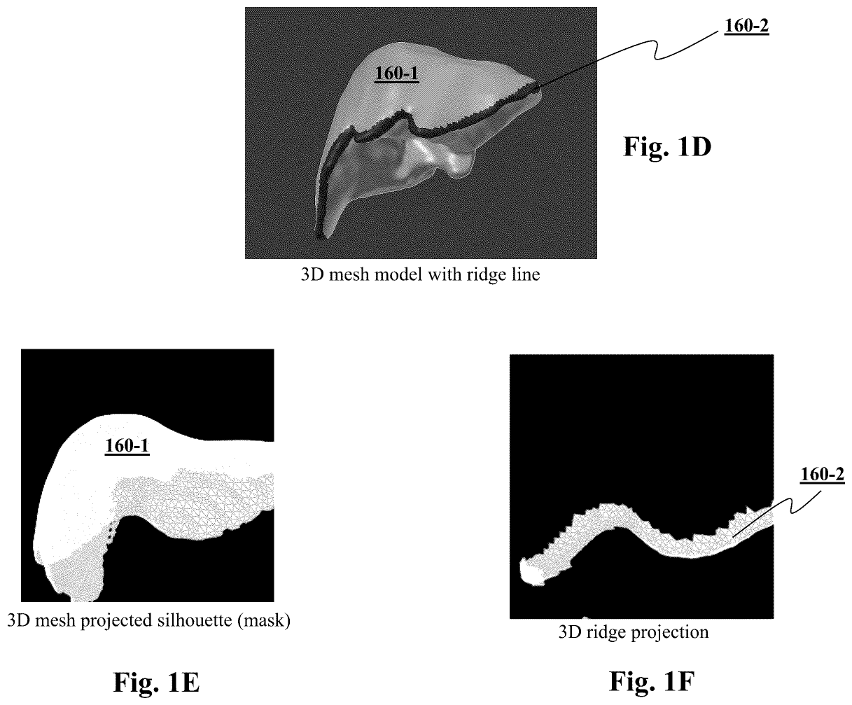

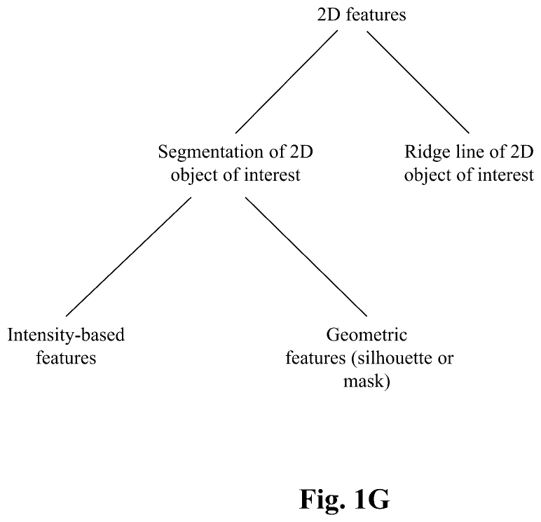

In some embodiments, different 3D camera poses may be assumed, each of which may be used to determine a perspective corresponding to the 3D camera pose which may be used to render the 3D model on a 2D plane to create a projection. 2D features of the projected 3D model as appearing on the 2D planes may be detected and leveraged to obtain mappings from 2D features to 3D camera poses. FIG. 1B illustrates an example 3D model 160 for an organ and an assumed 3D camera pose 170, which may be used to determine a perspective to look into the 3D model 160. FIG. 1C shows a 2D image plane 180 with a 2D projection 190 of the 3D model 160 onto the image plane 180 according to a perspective determined by the 3D camera pose 170. Such a projection 190 corresponds to a 2D structure with different features such as shape, size, etc. 2D features as they appear in the 2D image plane 180 may correspond to their 3D characteristics. FIG. 1D illustrates some exemplary 3D features of a liver modeled in the 3D model 160, including the 3D surface of the liver 160-1 as well as ridges 160-2. When the model 160 is projected onto a 2D image plane in a perspective perpendicular to FIG. 1D, these 3D features may be rendered in the 2D image plane as shown in FIGS. 1E (the surface of the liver model) and 1F (the ridges of the liver model).

As discussed herein, based on a 2D projection of a 3D model, a segmentation may be first obtained with respect to an object of interest, e.g., a liver. Based on the segmentation results, different 2D image features may be extracted from the segmented object. FIG. 1G illustrates exemplary types of 2D features that may be extracted from a projection of the 3D model, including features associated with the segmented object of interests such as intensity-related features (e.g., texture or color) within the segmented region and geometric features such as the silhouette or the shape of the segmented region. Some additional features may also be extracted from the segmented regions such as ridges along the surface of the liver. Such 2D features detected from a projected 2D image and the 3D camera pose that determines the perspective used for the projection may form a correspondence relation. When multiple 3D camera poses are used to generate corresponding 2D projections and 2D features extracted therefrom, the corresponding 3D camera poses and detected 2D features may be used to create a discrete mapping model between detected 2D features and 3D camera poses. Discrete mapping models may be constructed as lookup tables (LUTs) with 2D features and 3D camera poses pairings. Multiple LUTs may be constructed, each of which may be based on different 2D features. For instance, a LUT may be for mapping discretely 2D intensity features to 3D camera poses. Another LUT may be for mapping 2D geometric features such as shapes to poses. Yet another LUT may map a combination of 2D masks and ridge features to 3D camera poses. Although such discrete mappings are not continuous, in each case, it may identify the closest mapping through approximation. The estimated 3D camera poses obtained via approximation may optionally be refined or optimized to improve the precision of the 3D camera poses.

In a different embodiment, continuous mapping models may be constructed by using discrete 2D features/3D camera poses mappings as training data to train a mapping model, e.g., via machine learning, to learn complex relationships between 2D features and 3D camera poses so that the learned mapping models may be capable of mapping any given set of 2D features to some candidate 3D camera poses. Such a continuous mapping model may output multiple discrete outputs each of which may correspond to a 3D camera pose with a score, e.g., a probability, indicative of the confidence in the estimated 3D camera pose. In other embodiments, the multiple outputs of a trained mapping model may correspond to different degrees of freedom associated with 3D poses. Such a continuous mapping model may also have multiple outputs, each may relate to an estimated pose or a dimension parameter, with a corresponding confidence score associated therewith.

As discussed herein, different discreate mapping LUTs may be obtained, each of which may be based on a different type or combination of 2D features. This also applies to continuous mapping models. For example, a trained mapping model may be trained to map a combination of mask and ridge related 2D features to 3D camera poses. A different mapping model may be trained to map 2D geometric features of an object detected from 2D images to 3D poses. Yet another type of mapping model may be trained to map a combination of features (e.g., relating to intensity and geometric features to c3D camera poses. In each application scenario, an appropriate type of model (e.g., 2D mask and ridge feature-based mapping) may be invoked to estimate a 3D underlying camera pose. In some applications, more than one type of models (e.g., geometric shape, intensity, and ridge feature-based models) may be invoked to estimate the 3D pose and then the estimations from different models may be combined in some fashion to derive an overall estimate of the 3D camera pose. In some implementations, 2D features detected from 2D projections may be encoded so that the mappings between 2D features and 3D camera poses may be performed based on codes for 2D features. As the codes may be lighter weight as compared with 2D features such as masks or ridges, a mapping model trained based on codes may also be computationally more efficient so that the process of estimating 3D camera poses based on codes of 2D features may be carried out more efficiently. The encoding scheme to generate such codes for 2D features may be determined so that the 2D features may be reconstructed in a 2D image plane when needed.

As discussed herein, the output from a continuous mapping model may be a plurality of 3D camera poses each of which is associated with a score such as a probability indicating a confidence in the estimate. To determine a 3D camera pose, in some embodiments, the estimate with a top confidence score may be selected as the estimated 3D camera pose. Other implementations may also be possible to derive a final 3D camera pose estimate. In some embodiments, multiple (say, K) 3D camera pose estimates may be combined to generate an aggregated 3D pose estimate. For instance, top K estimates with, e.g., sufficient confidence scores may be aggregated in a weighted sum fashion to generate a final 3D camera pose estimate. In some situations, the weights applied to the individual estimates may be obtained according to their rankings determined according to, e.g., the confidence scores associated therewith. The aggregation may be performed by taking a weighted sum of the parameter in each dimension (each degree of freedom).

A 3D camera pose estimated based on 2D features such as masks and ridges according to the present teaching may be considered as an initial estimate and may optionally be further optimized or refined. According to the present teaching, in some embodiments, differential renderings may be used to facilitate the optimization. Based on an initial estimated 3D camera pose, the 3D model 160 may be rendered using slightly perturbed rendering parameters such as slightly displaced or rotated parameters to create differential projection results. In an iterative optimization process, each of the differential rendering results may be assessed against a loss function defined with respect to the pose parameters (e.g., 6-degrees of freedom) so that the 3D pose related parameters may be iteratively adjusted until convergence. The refined or optimized 3D camera pose estimate may then be used as the estimated 3D camera pose.

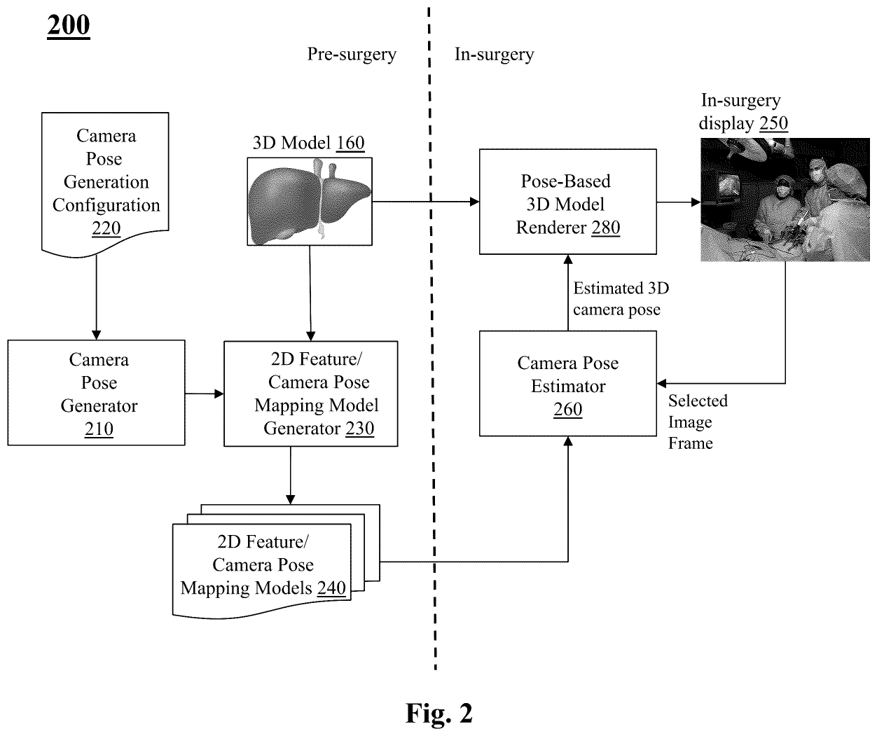

The trained 2D feature/3D camera pose mapping models, whether as a discrete LUT or in a continuous form, may be deployed in different applications. In one example, such models may be used in a laparoscopic procedure to operate on an organ to estimate the 3D pose of the laparoscopic camera based on 2D features extracted from laparoscopic images. The estimated 3D camera pose may then be used, in conjunction with a 3D model for the organ, to determine a perspective to project the 3D model onto a display to provide a 3D visual guide that is aligned with what is seen in the 2D laparoscopic images. In some embodiments, the display may correspond to superimposed 3D projection of the 3D model. In some embodiments, a separate screen may be rendered with the projected 3D model side by side with the laparoscopic image. Such a projection of a 3D model may also include different anatomical structures beneath the surface of the organ, so that the projection of the 3D model in an aligned manner provides an effective visual assistance to a surgeon in a laparoscopic procedure. Details related to the present teaching on estimating a 3D camera pose based on 2D image features are provided below with reference to FIGS. 2-FIG. 6B.

FIG. 2 depicts an exemplary high level system diagram of a 3D camera pose estimation and use case framework 200, in accordance with an embodiment of the present teaching. Framework 200 includes two portions, one being a pre-surgery portion and the other being an in-surgery portion. The pre-surgery portion is provided for establishing 2D feature/camera pose mapping models 240 and includes a camera pose generator 210 and a 2D feature/camera pose mapping model generator 230. In this illustrated embodiment, the camera pose generator 210 is provided for generating a series of assumed 3D camera poses based on camera pose generation configuration 220 (which may specify, e.g., the resolution used to generate the 3D camera poses). The generated 3D camera poses may then be provided to the 2D feature/camera pose mapping model generator 230, which may utilize the series of assumed 3D camera poses to generate the 2D feature/camera pose mapping models with respect to a 3D model 160 for an organ.

Specifically, as discussed herein, to create the mapping models 240, for each of the assumed 3D camera poses generated by the camera pose generator 210, a corresponding viewing perspective is determined (see the example view perspective determined based on 3D camera pose 170 in FIG. 1B) and used to project the 3D model 160 onto a 2D image plane, such as the one shown in FIG. 1C. Based on the projection, 2D features are extracted (e.g., a 2D mask for the object of interest and ridges thereon) and are then used to pair with the 3D camera pose used to make the projection. In this manner, pairs of 2D features and 3D camera poses are created and are used to derive the 2D feature/3D camera pose mapping models 240. As discussed herein, in some embodiments, the pairs may be used to build discrete models as lookup tables (LUTs). This may work well when the resolution used to generate the 3D camera poses is sufficiently fine so that the 3D camera poses estimated via the LUT is relatively accurate. In other embodiments, the created pairs of 2D features and 3D camera poses may be used as training data for training in a machine learning process to obtain continuous mapping models as discussed herein. Such obtained mapping models 240 may then be used by the in-surgery part to estimate 3D camera poses based on 2D featured extracted from 2D images acquired during a surgery.

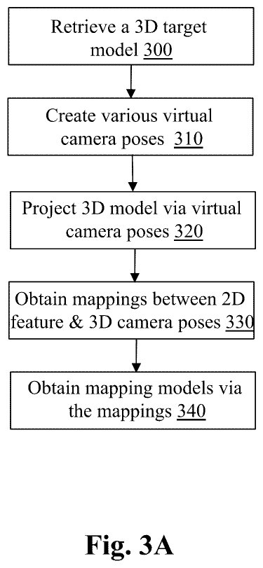

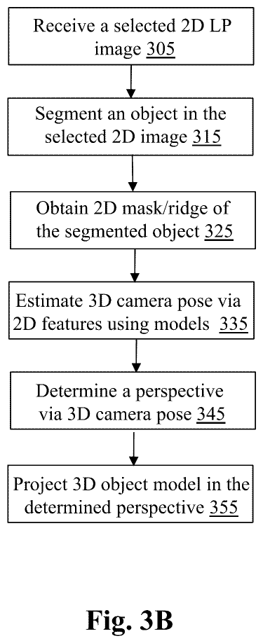

FIG. 3A is a flowchart of an exemplary process of the first part of framework 200 for deriving models for mapping 2D features to a 3D camera pose, in accordance with an embodiment of the present teaching. A 3D model 160 for a relevant organ is first retrieved at 300. To generate mapping models, the camera pose generator 210 generates, at 315, various virtual 3D camera poses according to some generation parameters (e.g., resolution, etc.) specified in configuration 220. Such generated virtual 3D camera poses are provided to the 2D feature/camera pose mapping model generator 230, which projects, at 320, the 3D model 160 in different perspectives determined according to the virtual 3D camera poses to obtain corresponding 2D projected images. 2D features may then be extracted, at 325, from each of the 2D projected images and used, with respect to the corresponding virtual 3D camera pose, to create a pair or mapping, at 330, between 2D features and each corresponding 3D camera pose. Such created mappings corresponding to discrete pairs and may be used to derive either discrete or continuous mapping models. In operation, depending on the types of models to be created (e.g., discrete or continuous, the operation of deriving the 2D feature/camera pose mapping model generator 230 may differ. For instance, the mappings created between virtual 3D camera poses and the extracted 2D features may be used directly as a discrete mapping model. On the other hand, to derive continuous mapping models, such discrete mappings may be used as training data for machine learning of respective continuous mapping models.

The second portion of the framework 200 is to apply such derived mapping models for estimating 3D camera poses based on 2D features identified from 2D images acquired during a medical procedure. In some embodiments, the acquired 2D images may correspond to laparoscopic images obtained via a laparoscopic camera and the task is to estimate the 3D pose of the laparoscopic camera. The second portion of the framework 200, as shown in FIG. 2, includes an in-surgery display 250, a camera pose estimator 260, and a pose-based 3D model renderer 270. The in-surgery display 250 is provided in a surgery room for displaying 2D images acquired by a laparoscopic camera inserted into a patient's body, such as camera 130 illustrated in FIG. 1A. One of the acquired 2D images may be selected and used by the are processed by the camera pose estimator 260 to segment an object of interest, e.g., an organ such as a liver and 2D features thereof for estimating the 3D pose of the camera. In some embodiments, when the laparoscopic camera acquires a stream of 2D images (video), via interaction with a surgeon, a particular 2D image may be identified as the selected 2D image for estimating a corresponding 3D camera pose. For example, a laparoscopic image with satisfactory quality may be selected for estimating the 3D camera pose.

The camera pose estimator 260 is provided for estimating a 3D camera pose with respect to the selected 2D image based on the 2D feature/camera pose mapping models 240, according to the present teaching. With the estimated 3D camera pose from the camera pose estimator 260, the pose-based 3D model renderer 270 is provided to use the estimated 3D camera pose to determine a perspective for projecting the 3D model 160 of the organ at issue. This creates a 3D rendering of the model 160 that is in alignment with the selected laparoscopic image. The rendered 3D model 160 provides a more effective visual guidance to a surgeon not only because it aligns with the laparoscopic image but also because it reveals the anatomical structures inside of the organ which is otherwise invisible from the 2D laparoscopic images. As discussed herein, the 3D model 160 may be rendered by superimposing on the 2D images. In other embodiments, the 3D model 160 may be rendered on a separate display, e.g., either a different display window of the same display screen where the 2D images are displayed or on a different display device. Such a rendering may be displayed side-by-side with the 2D images.

FIG. 3B is a flowchart of an exemplary process of the second part of framework 200 for leveraging the 2D feature/camera pose mapping models 240 during a surgery to estimate a 3D laparoscopic camera pose for aligning a 3D model with the laparoscopic images, in accordance with an embodiment of the present teaching. In a laparoscopic procedure, the camera pose estimator 260 first receives, at 305, a selection of a 2D laparoscopic image. The selected laparoscopic image is then processed to segment, at 315, an object (e.g., a liver) of interest therefrom. Based on the segmentation result, a 2D mask and ridges associated with the segmented object may be obtained at 325 and are used as 2D features to estimate, at 335, the 3D pose of the laparoscopic camera based on the 2D feature/camera pose mapping models 240. As discussed herein, in some embodiments, the 2D feature/camera pose mapping models 240 may be trained to take codes of the 2D features as input. In this case, the 2D features (including mask and ridges) may first be encoded to generate a code which is then provided to the mapping model 240 as the input to obtain the estimated 3D camera pose.

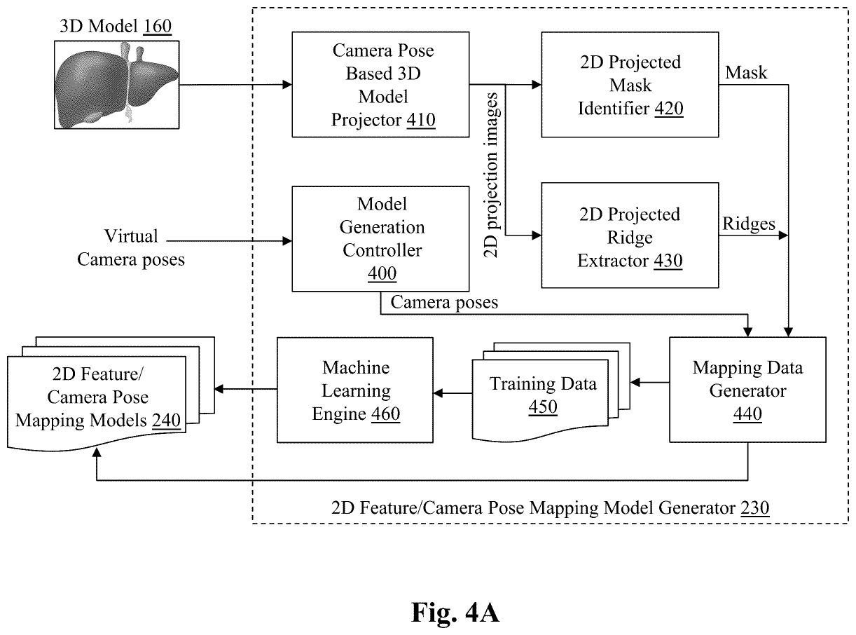

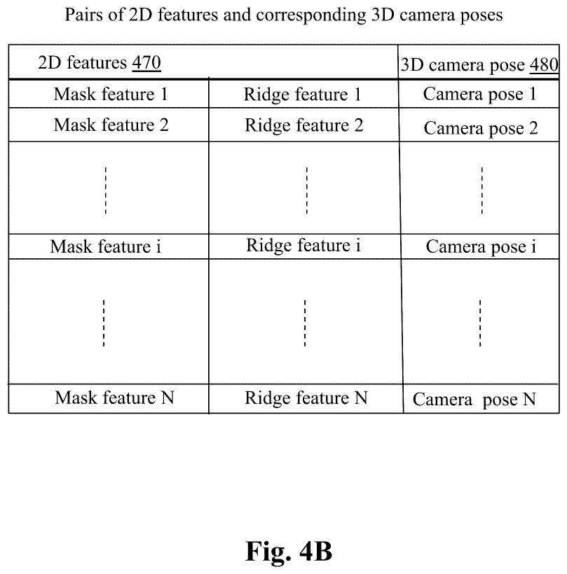

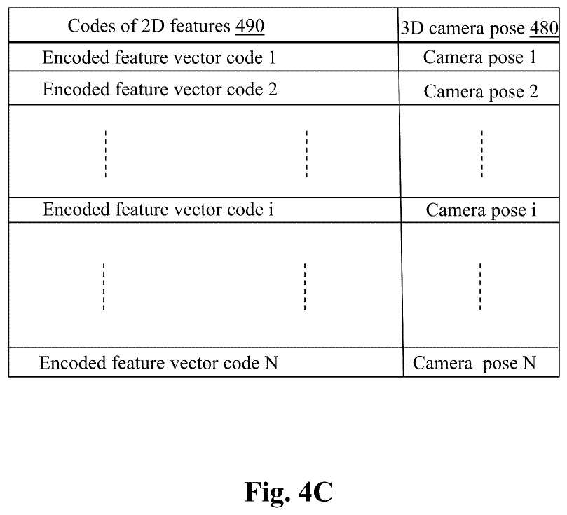

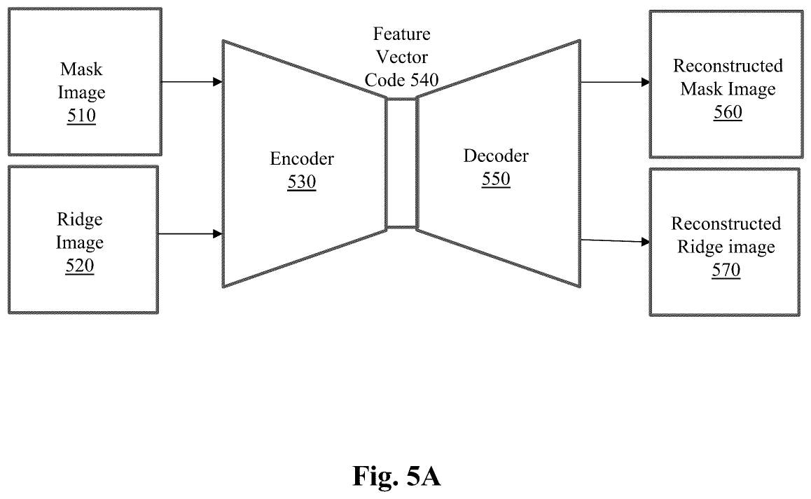

As discussed herein, depending on the type of the mapping models, an initial 3D camera pose may be generated as the output of the camera pose estimator 260. In some embodiments, as an alternative, the camera pose estimator 260 may optionally further optimize the initial 3D camera pose estimate to produce an optimized estimated 3D camera pose. The 3D camera pose estimate from the camera pose estimator 260 (either initial or optimized) may then be used by the pose-based 3D model renderer 270 to determine, at 345, a rendering perspective based on the estimated 3D camera pose and then project, at 355, the 3D model 160 of the organ on a display according to the perspective. Details related to the camera pose estimator 270 are provided below with reference to FIGS. 4A-6B.

FIG. 4A depicts an exemplary high level system diagram of the 2D feature/camera pose mapping model generator 230, in accordance with an embodiment of the present teaching. As discussed herein, the 2D feature/camera pose mapping model generator 230 is provided for creating mapping models 240 between 2D features detected from laparoscopic images or codes encoded therefor and 3D camera poses via virtual 2D images created in different perspectives corresponding to assumed 3D camera poses. The generator 230 may take virtual camera poses (from the camera pose generator 210) as input and generate the 2D feature/camera pose mapping models 240. This illustrated embodiment is provided for deriving either discrete or continuous mapping models and comprises a model generation controller 400, a camera-pose based 3D model projector 410, a 2D projected mask identifier 420, a 2D projected ridge extractor 430, a mapping data generator 440, and a machine learning engine 460.

The model generation controller 400 is provided for taking input virtual camera poses as input and accordingly control the operation of generating the 2D feature/camera pose mapping models 240. Based on each input virtual 3D camera pose, the camera-pose based 3D model projector 410 is invoked to determine a corresponding projection perspective based on the input virtual camera pose and then project the 3D model 160 according to the corresponding perspective to generate a 2D virtual projection image. Such a virtual projection image may then be used by the 2D projected mask identifier 420 to identify a mask for the object of interest (e.g., a liver) and by the 2D projected ridge extractor 430 to extract the ridges present therein. The mapping data generator 440 may be provided to take the camera pose as well as the 2D features (mask and ridges) detected from the projected 2D image as input and form the pairing between the 2D features and the 3D camera pose. Based on multiple input virtual camera poses and the corresponding 2D features extracted from the 2D virtual images projected accordingly, mappings may be formed based on such multiple pairings.

FIG. 4B illustrates exemplary pairings of 2D features and camera poses, in accordance with an embodiment of the present teaching. As shown in FIG. 4B, each pairing corresponds to one row which includes 2D features 470 and 3D camera pose 480. In the illustration shown in FIG. 4B, each 3D camera pose may be represented as a tuple with 6 parameters (X, Y, Z, p, r, y), corresponding to six degrees of freedom, including (X, Y, Z) representing a coordinate in a 3D camera space, and (p, r, y) representing an orientation measured in terms of pitch (p), roll (r), and yaw (y), respectively. In some embodiments, the 2D features may be encoded so that the pairings may be between the 2D feature codes 490 with 3D camera poses 480, as illustrated in FIG. 4C according to an embodiment of the present teaching. In this illustration, 2D features (e.g., mask and ridges) detected from a 2D image may be combined into a feature vector which may be encoded to generate a code and then paired with a corresponding 3D camera pose. Using such encoded 2D features may make the 2D feature/camera pose mapping models 240 more efficient because there is no need to use images with 2D features for paring with the 3D camera poses. This is especially so when the mapping models 240 are constructed based on millions of pairings.

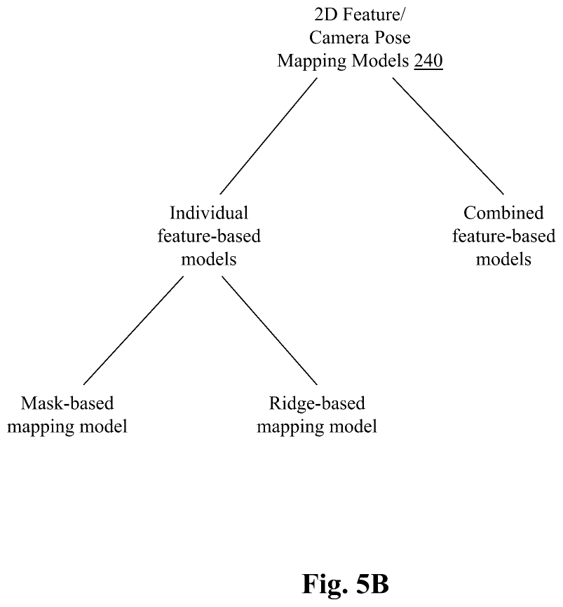

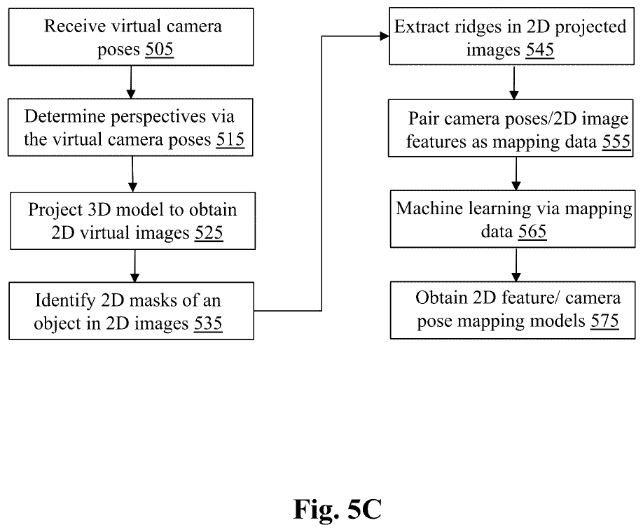

In some embodiments, in some situations 2D features may need to be reconstructed by decoding a code. For example, the mapping models 240 may produce multiple candidate camera pose estimates with different confidence levels and to select one from such initial output, the 2D features may be needed in order to facilitate the selection. An appropriate encoding scheme may be used for encoding 2D features so that the underlying 2D features may be effectively reconstructed based on a code. FIG. 5A depicts an exemplary process for encoding 2D features to generate a code when building the mapping model 240 and when needed, the code may be used to reconstruct, via decoding, the 2D features. As shown, a mask image 510 and a ridge image 520 may be processed by an encoder 530 to generate a feature vector code 540 to efficiently represent the 2D features 510 and 520. Such a code 540 may be, when needed, decoded by a decoder 550 to generate a reconstructed mask image 560 and a reconstructed ridge image 570. In some embodiments, a reconstructed image, e.g., reconstructed mask image 560, may be used to, e.g., compare with a mask image obtained based on a laparoscopic image to assess the similarity.

The pairings between 2D features (or codes thereof) and 3D camera poses may be used directly as a LUT as a discrete mapping model. In this case, the mappings created by the mapping data generator 440 based on the parings may be stored as the 2D feature/camera pose mapping models 240. In some embodiments, these paired mappings may be used as training data 450 for machine learning by the machine learning engine 460 to obtain continuous 2D feature/camera pose mapping models 240.

Although 2D features mask and ridge are disclosed herein as an illustration, other 2D features may also be used to pair with the 3D camera poses to derive mapping models 240 as needed for any application in hand. FIG. 5B illustrates different 2D feature/camera pose mapping models that may be derived based on 2D features, in accordance with an embodiment of the present teaching. For instance, mapping models may be obtained based on individual types of 2D features, to create, e.g., separate mask-based mapping model or ridge-based mapping model, either a discrete or a continuous model. A mapping model may also be obtained based on a combination of different 2D features (e.g., both mask and ridges).

FIG. 5C is a flowchart of an exemplary process for the 2D feature/camera pose mapping model generator 230, in accordance with an embodiment of the present teaching. In operation, when input virtual 3D camera poses are received at 505, the mode generation controller 400 send the camera poses to the camera pose based 3D model projector 410 which determines, at 515, the corresponding perspectives for the projection and accordingly projects, at 525, the 3D model 160 to generate 2D projected virtual images. The 2D projected mask identifier 420 may then identify, at 535, masks of the object of interest in the 2D projected virtual images and the 2D projected ridge extractor 430 may also extract, at 545, ridges of the object of interest from the 2D projected virtual images. The obtained masks and ridges may then be paired, at 555, with corresponding 3D camera poses by the mapping data generator 440. Depending on the operational mode controlled by the model generation controller 400, such generated mapping data may be stored in 240 directly as a discrete mapping model. If the operation mode is to generate a continuous mapping model, the mapping data generated by generator 440 may then be stored as training data in 450 (see FIG. 4A) which may then be used by the machine learning engine 460 to learn, at 565, via machine learning to obtain, at 575, a continuous 2D feature/camera pose mapping model 240.

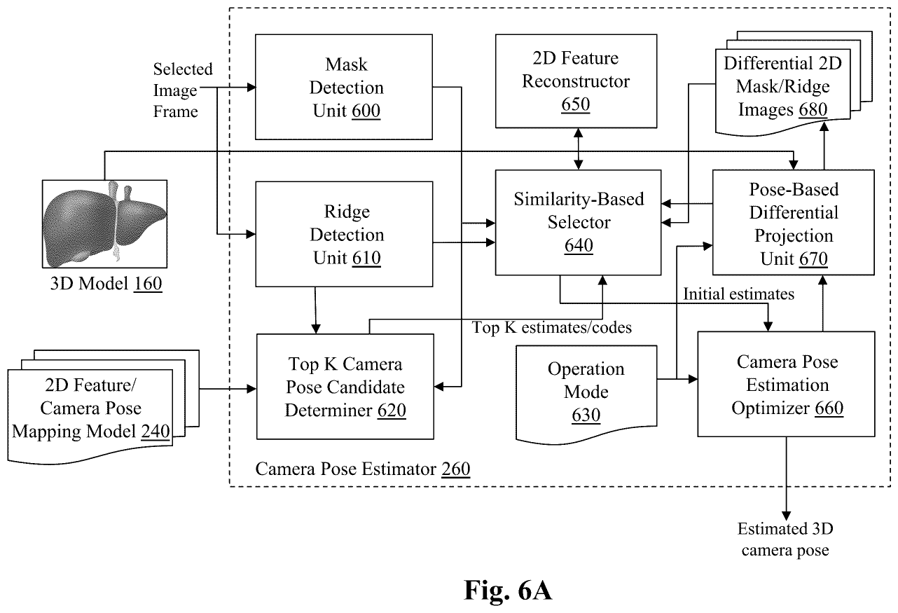

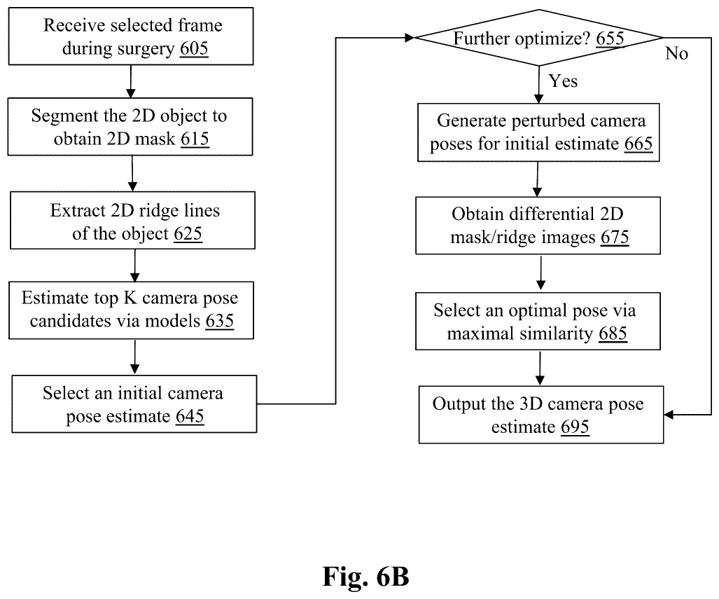

FIG. 6A depicts an exemplary high level system diagram of the camera pose estimator 260, in accordance with an embodiment of the present teaching. In this illustrated embodiment, there may include two parts. The first part may be provided for estimating an initial 3D camera pose based on 2D mask and ridge features. The second part may be provided as an option to optimize the initial estimated 3D camera pose to derive a refined 3D camera pose estimation. The first part may comprise a mask detection unit 600, a ridge detection unit 610, a top K camera pose candidate determiner 620, and a similarity-based selector 640, and optionally a 2D feature reconstructor 650.

Given an input laparoscopic image, the mask detection unit 600 and the ridge detection unit 610 are used to identify a mask of an object of interest and extract ridges associated with the object, respectively. These detected 2D features are then used by the top K camera pose candidate determiner 620 to estimate top K camera pose candidates based on the 2D feature/camera pose mapping models 240. In some embodiments, when the 2D feature/camera pose mapping models 240 correspond to LUTs, the top K camera pose candidates may be identified based on best matches of 2D features. For example, top 5 camera poses may be obtained by selecting 5 rows that yield the closest matches to the detected 2D features. If the mapping models 240 are continuous models, the top K camera pose candidates may correspond to those with the top 5 highest ranked confidence levels. In some embodiments, if the 2D feature/camera pose mapping models 240 operate based on encoded 2D features (i.e., codes), the top K camera pose candidate determiner 620 may first encode the detected 2D features to obtain a code and then operate on the code to derive estimated 3D camera pose candidates based on the mapping models 240.

The top K candidate camera poses may provide a range of choices from which to identify one as an initial 3D camera pose estimate. To achieve that, the top K camera pose candidates with the corresponding top K 2D feature sets (or their codes), may be provided to the similarity-based selector 630 for the selection. In some embodiments, this may be achieved by comparing the 2D features detected from the given input laparoscopic image with the 2D features from the mapping model 240. When the 2D features represented by the mapping models 240 are codes, the 2D feature reconstructor 650 may first be invoked to reconstruct 2D features based on the codes prior to being compared with the 2D featured detected from the laparoscopic image. Via comparison, the camera pose estimate that pairs with 2D features that are best matched with the 2D features detected from laparoscopic image may then be selected as the initial 3D camera pose estimate. The first part of the camera pose estimator 260 outputs the initial camera pose estimate.

As discussed herein, the second part of the camera pose estimator 260 may be optionally provided to refine the initial camera pose estimate to generate an optimized 3D camera pose estimate. The second part comprises a camera pose estimation optimizer 660, a pose-based differential projection unit 670, and the similarity-based selector 640. In some embodiments, the operation of the camera pose estimation optimizer 660 may be controlled according to the operation mode specified in 630. In some situations, the operation mode 630 may be configured as no optimization so that the initial camera pose estimate selected from the top K candidates may be directly output as the estimated 3D camera pose.

When the operation mode 630 is configured to further optimize the initial 3D camera pose estimate, further optimization may be performed based on optimization parameters specified in 630. In some embodiments, the optimization may be based on differential projection using perturbed pose parameters (with respect to different degrees of freedom, including the coordinate, its pitch, roll, and yaw) and similarity-based 2D feature comparison. Based on the initial camera pose estimate, the camera pose estimation optimizer 660 may be provided to generate perturbed camera poses according to the optimization parameters specified in 630 (e.g., the scope and resolution of the perturbation with respect to each degree of freedom). The pose-based differential projection unit 670 may be invoked to create differential 2D mask/ridge images via differential projections of the 3D model 160 using the perturbed camera poses. In some embodiments, an optimization scheme may be deployed that may, based on the differential 2D mask/ridge images, select an optimal perturbed 3D camera pose corresponding to a differential 2D mask/ridge image that yields, e.g., a maximal similarity (assessed by, e.g., the similarity-based selector 540) with that extracted from an input laparoscopic image.

FIG. 6B is a flowchart of an exemplary process of the camera pose estimator 260, in accordance with an embodiment of the present teaching. In operation, when the input 2D laparoscopic image is received at 605, the mask detection unit 600 segments, at 615, the 2D laparoscopic image to identify the mask corresponding to an object of interest (e.g., a liver) and the ridge detection unit 610 extracts, at 625, ridge lines associated with the object of interest. Based on the obtained 2D mask/ridges, the top K camera pose candidate determiner 620 estimates, at 635, top K camera pose estimates based on the 2D feature/camera pose mapping models 240. As discussed herein, in some embodiments, the 2D features used for mapping may be first encoded as a code (when the 2D feature/camera pose mapping models 240 are constructed based on encoded 2D features) and then used to obtain the top K candidates via the 2D feature/camera pose mapping models 240 (e.g., a LUT or a continuous model).

From the top K camera pose candidates, the similarity-based selector 640 may select, at 645, an initial camera pose estimate based on the similarity between the 2D features of the top K camera pose candidates and that extracted from the input laparoscopic image. In some embodiments, if the mapping models 240 are constructed using encoded 2D features (i.e., codes), the 2D feature reconstructor 650 may be invoked first to decode the codes of the top K camera pose candidates to obtain the reconstructed 2D features for the top K candidates, which are then used for evaluating the similarity with that of the input laparoscopic image. According to the operational mode configured in 630, if no additional optimization is needed, determined at 655, the selected initial camera pose is output, at 695, as the estimated 3D camera pose for the laparoscopic camera. Otherwise, the camera pose estimation optimizer 660 may proceed with the further optimization by first generating, at 665, perturbed camera poses based on the initial camera pose estimate in accordance with the operation mode configuration 630 (specifying, e.g., perturbation scope and resolutions in different dimensions) which are then used by the pose-based differential projection unit 670 to obtain, at 675, differential 2D mask/ridge images. This may be achieved by projecting the 3D model 160 using perspectives determined based on the perturbed camera poses. 2D features for such differential 2D mask/ridge images may then be evaluated in terms of their similarities to that of the input laparoscopic image and one of the perturbed camera poses may be selected, at 685, as the optimal 3D camera pose estimate when its corresponding differential 2D mask/ridge image yields a maximal similarity with that of the input laparoscopic image. Such an optimized 3D camera pose estimate may then be output at 695.

As shown in FIG. 2A, the estimated 3D camera pose may be used by the pose-based 3D model renderer 280 to render the 3D model 160 to provide an effective visual guidance to a user during a medical procedure. Depending on the application needs, the pose-based 3D model renderer 280 may be configured to render the 3D model 160 in different ways. In some situations, distinct stages of the same procedure may be configured differently to render the 3D model 160 to show different types of information. For instance, to remove a tumor inside of a liver, a surgeon may first clamp some major blood vessels connected to the liver to prevent blood loss when cutting open the liver to remove the tumor. Once the outside blood vessels are clamped, the surgeon may need to see the inside anatomical structures including the tumor and the blood vessels that supply the blood to the tumor. At this point, the surgeon may desire to see what is beneath the surface of the liver via 3D model rendering so that the 3D model 160 may be rendered to show all anatomical structures and their spatial relationships to assist the surgeon how to manipulate a surgical instrument.

As such, the pose-based 3D model renderer 280 may be configured to render the 3D model 160 based on estimated 3D camera pose according to the needs during different stages of a laparoscopic procedure. For example, when a surgical instrument is still in the process of approaching an object of interest such as a liver, the 3D model 160 may be rendered to show the liver in terms of its physical properties (e.g., shape and size) and its nearby anatomical structures such as nearby blood vessels or bones. Such rendered information may assist a surgeon to, e.g., clamp some blood vessels to stop the blood supply to, e.g., a tumor inside of a liver before removal of the tumor. Once the surgeon is ready to remove a tumor insider a liver, the 3D model 160 may be rendered to provide a visual guidance as to what is beneath the surface of an object of interest such as a liver, e.g., the location of a tumor and blood vessels connected to the tumor to allow the surgeon to perform what they need to.

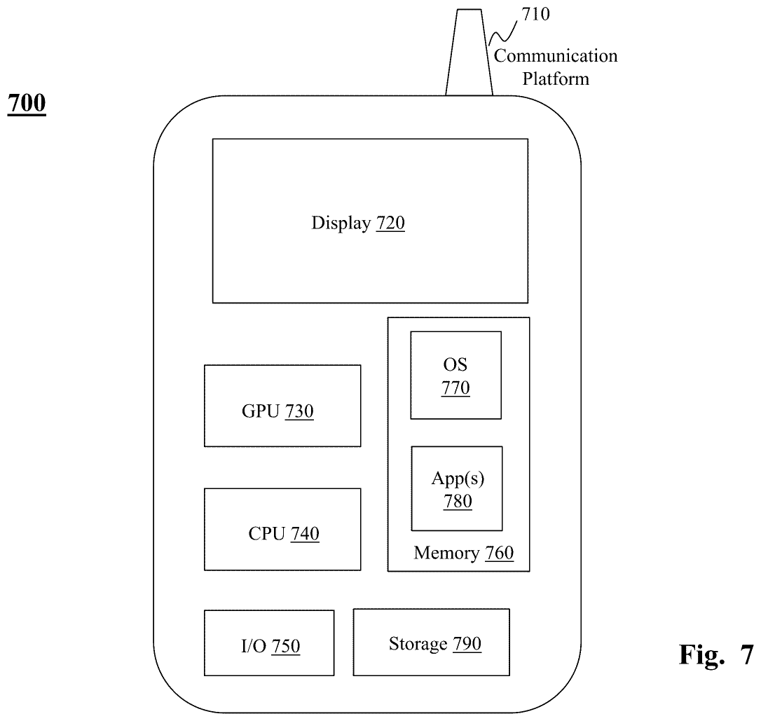

FIG. 7 is an illustrative diagram of an exemplary mobile device architecture that may be used to realize a specialized system implementing the present teaching in accordance with various embodiments. In this example, the user device on which the present teaching may be implemented corresponds to a mobile device 700, including, but not limited to, a smart phone, a tablet, a music player, a handled gaming console, a global positioning system (GPS) receiver, and a wearable computing device, or in any other form factor. Mobile device 700 may include one or more central processing units (“CPUs”) 740, one or more graphic processing units (“GPUs”) 730, a display 720, a memory 760, a communication platform 710, such as a wireless communication module, storage 790, and one or more input/output (I/O) devices 750. Any other suitable component, including but not limited to a system bus or a controller (not shown), may also be included in the mobile device 700. As shown in FIG. 7, a mobile operating system 770 (e.g., iOS, Android, Windows Phone, etc.), and one or more applications 780 may be loaded into memory 760 from storage 790 to be executed by the CPU 740. The applications 780 may include a user interface or any other suitable mobile apps for information analytics and management according to the present teaching on, at least partially, the mobile device 700. User interactions, if any, may be achieved via the I/O devices 750 and provided to the various components connected via network(s).

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar with to adapt those technologies to appropriate settings as described herein. A computer with user interface elements may be used to implement a personal computer (PC) or other type of workstation or terminal device, although a computer may also act as a server if appropriately programmed. It is believed that those skilled in the art are familiar with the structure, programming, and general operation of such computer equipment and as a result the drawings should be self-explanatory.

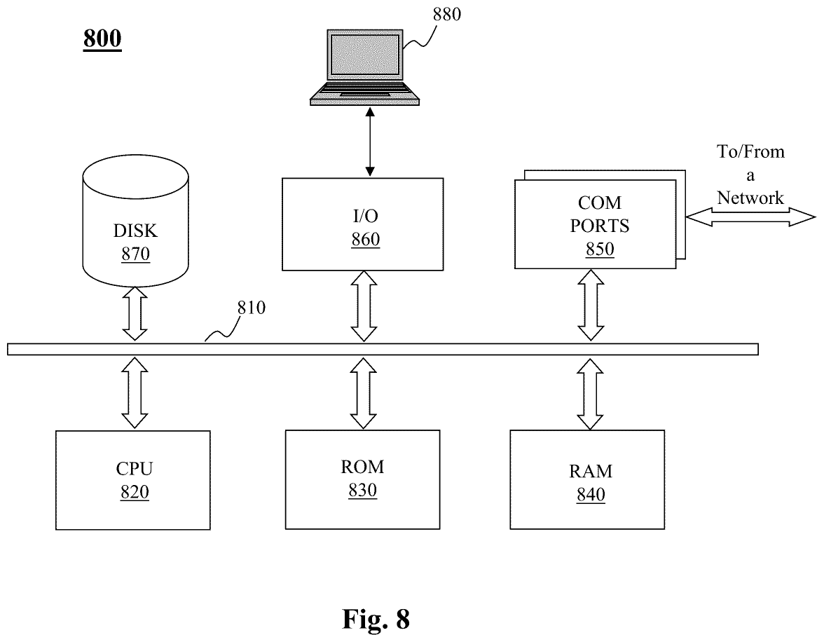

FIG. 8 is an illustrative diagram of an exemplary computing device architecture that may be used to realize a specialized system implementing the present teaching in accordance with various embodiments. Such a specialized system incorporating the present teaching has a functional block diagram illustration of a hardware platform, which includes user interface elements. The computer may be a general-purpose computer or a special purpose computer. Both can be used to implement a specialized system for the present teaching. This computer 800 may be used to implement any component or aspect of the framework as disclosed herein. For example, the information analytical and management method and system as disclosed herein may be implemented on a computer such as computer 800, via its hardware, software program, firmware, or a combination thereof. Although only one such computer is shown, for convenience, the computer functions relating to the present teaching as described herein may be implemented in a distributed fashion on a number of similar platforms, to distribute the processing load.

Computer 800, for example, includes COM ports 850 connected to and from a network connected thereto to facilitate data communications. Computer 800 also includes a central processing unit (CPU) 820, in the form of one or more processors, for executing program instructions. The exemplary computer platform includes an internal communication bus 810, program storage and data storage of different forms (e.g., disk 870, read only memory (ROM) 830, or random-access memory (RAM) 840), for various data files to be processed and/or communicated by computer 800, as well as possibly program instructions to be executed by CPU 820. Computer 800 also includes an I/O component 860, supporting input/output flows between the computer and other components therein such as user interface elements 880. Computer 800 may also receive programming and data via network communications.

Hence, aspects of the methods of information analytics and management and/or other processes, as outlined above, may be embodied in programming. Program aspects of the technology may be thought of as “products” or “articles of manufacture” typically in the form of executable code and/or associated data that is carried on or embodied in a type of machine-readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable loading of the software from one computer or processor into another, for example, in connection with information analytics and management. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as a main memory of such a computer platform. Tangible transmission media include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described above may be embodied in a hardware device, it may also be implemented as a software only solution, e.g., an installation on an existing server. In addition, the techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

We claim:

1. A method comprising:
generating virtual 3D camera poses with respect to a 3D model previously constructed to model a 3D target organ and 3D anatomical structures associated therewith, wherein each of the virtual 3D camera poses corresponds to a perspective to view the 3D model;
creating virtual 2D images corresponding to the virtual 3D camera poses by projecting the 3D model in accordance with corresponding perspectives, wherein each of the virtual 2D images includes 2D projected target organ and/or 2D structures of some of the 3D anatomical structures visible from a corresponding perspective; and
obtaining 2D feature/camera pose mapping models based on the 2D features extracted from the virtual 2D images and the corresponding virtual 3D camera poses, wherein the 2D features include a 2D ridge line projected from a 3D ridge on the target organ represented in the 3D model.

2. The method of claim 1, wherein the 3D model models at least one of:
the target organ,
at least one blood vessel;
at least one tumor; and
one or more 3D ridges on the target organ.

3. The method of claim 1, wherein
each of the virtual 3D camera poses is characterized in terms of six-degrees of freedom; and
the virtual 3D camera poses are generated to cover different viewing angles with respect to the 3D model with an increment in each of the six-degrees of freedom according to a pre-determined resolution.

4. The method of claim 1, wherein the 2D features extracted from each of the virtual 2D images include one or more of:
a 2D structure corresponding to a 2D projection of the target organ in the virtual 2D image;
a mask of the 2D structure corresponding to the target organ;
a 2D ridge projected from a 3D ridge on the target organ modeled by the 3D model.

5. The method of claim 1, wherein the step of obtaining 2D feature/camera pose mapping models comprises:
pairing each of the virtual 3D camera poses with 2D features extracted from a corresponding virtual 2D image created by projecting the 3D model in accordance with a perspective determined based on the virtual 3D camera pose; and
creating the 2D feature/camera pose mapping models based on the pairs of the 2D features and the virtual 3D camera poses.

6. The method of claim 5, wherein the 2D feature/camera pose mapping models correspond to a look-up table comprising the pairs of the 2D features and the virtual 3D camera poses so that given input 2D features extracted from a 2D image, at least one 3D camera pose is identified from a pair in the look-up table that has stored 2D features similar to the input 2D features.

7. The method of claim 5, wherein the step of creating the 2D feature/camera pose mapping tools comprises:
generating training data based on the pairs of the 2D features and the virtual 3D camera poses;
performing machine learning, using the training data, to learn the 2D feature/camera pose mapping tools.

8. The method of claim 1, further comprising:
receiving, during a medical procedure, a 2D image acquired by a camera inserted into a patient's body near the target object to capture surrounding information;
detecting, from the 2D image, a 2D object corresponding to the target organ and/or 2D structures corresponding to some of the 3D anatomical structures;
extracting 2D features of the detected 2D object and/or 2D structures;
predicting, based on the 2D feature/camera pose mapping models, an estimated 3D camera pose of the camera; and
projecting the 3D model to visualize the target organ and/or some of the anatomical structures associated therewith in accordance with a perspective determined based on the estimated 3D camera pose.

9. A machine-readable medium having information recorded thereon, wherein the information, when read by the machine, causes the machine to perform the following steps:
generating virtual 3D camera poses with respect to a 3D model previously constructed to model a 3D target organ and 3D anatomical structures associated therewith, wherein each of the virtual 3D camera poses corresponds to a perspective to view the 3D model;
creating virtual 2D images corresponding to the virtual 3D camera poses by projecting the 3D model in accordance with corresponding perspectives, wherein each of the virtual 2D images includes 2D projected target organ and/or 2D structures of some of the 3D anatomical structures visible from a corresponding perspective; and
obtaining 2D feature/camera pose mapping models based on the 2D features extracted from the virtual 2D images and the corresponding virtual 3D camera poses, wherein the 2D features include a 2D ridge line projected from a 3D ridge on the target organ represented in the 3D model.

10. The medium of claim 9, wherein the 3D model models at least one of:
the target organ,
at least one blood vessel;
at least one tumor; and
one or more 3D ridges on the target organ.

11. The medium of claim 9, wherein
each of the virtual 3D camera poses is characterized in terms of six-degrees of freedom; and
the virtual 3D camera poses are generated to cover different viewing angles with respect to the 3D model with an increment in each of the six-degrees of freedom according to a pre-determined resolution.

12. The medium of claim 9, wherein the 2D features extracted from each of the virtual 2D images include one or more of:
a 2D structure corresponding to a 2D projection of the target organ in the virtual 2D image;
a mask of the 2D structure corresponding to the target organ;
a 2D ridge projected from a 3D ridge on the target organ modeled by the 3D model.

13. The medium of claim 9, wherein the step of obtaining 2D feature/camera pose mapping models comprises:
pairing each of the virtual 3D camera poses with 2D features extracted from a corresponding virtual 2D image created by projecting the 3D model in accordance with a perspective determined based on the virtual 3D camera pose; and
creating the 2D feature/camera pose mapping models based on the pairs of the 2D features and the virtual 3D camera poses.

14. The medium of claim 13, wherein the 2D feature/camera pose mapping models correspond to a look-up table comprising the pairs of the 2D features and the virtual 3D camera poses so that given input 2D features extracted from a 2D image, at least one 3D camera pose is identified from a pair in the look-up table that has stored 2D features similar to the input 2D features.

15. The medium of claim 13, wherein the step of creating the 2D feature/camera pose mapping tools comprises:
generating training data based on the pairs of the 2D features and the virtual 3D camera poses;
performing machine learning, using the training data, to learn the 2D feature/camera pose mapping tools.

16. The medium of claim 9, wherein the information, when read by the machine, further causes the machine to perform the following steps:
receiving, during a medical procedure, a 2D image acquired by a camera inserted into a patient's body near the target object to capture surrounding information;
detecting, from the 2D image, a 2D object corresponding to the target organ and/or 2D structures corresponding to some of the 3D anatomical structures;
extracting 2D features of the detected 2D object and/or 2D structures;
predicting, based on the 2D feature/camera pose mapping models, an estimated 3D camera pose of the camera; and
projecting the 3D model to visualize the target organ and/or some of the anatomical structures associated therewith in accordance with a perspective determined based on the estimated 3D camera pose.

17. A system comprising:
a camera pose generator implemented by a processor and configured for generating virtual 3D camera poses with respect to a three-dimensional (3D) model previously constructed to model a 3D target organ and 3D anatomical structures associated therewith, wherein each of the virtual 3D camera poses corresponds to a perspective to view the 3D model;
a 2D feature/camera pose mapping model generator implemented by a processor and configured for
creating virtual 2D images corresponding to the virtual 3D camera poses by projecting the 3D model in accordance with corresponding perspectives, wherein each of the virtual 2D images includes 2D projected target organ and/or 2D structures of some of the 3D anatomical structures visible from a corresponding perspective, and
obtaining 2D feature/camera pose mapping models based on the 2D features extracted from the virtual 2D images and the corresponding virtual 3D camera poses, wherein the 2D features include a 2D ridge line projected from a 3D ridge on the target organ represented in the 3D model.

18. The system of claim 17, wherein the 3D model models at least one of:
the target organ,
at least one blood vessel;
at least one tumor; and
one or more 3D ridges on the target organ.

19. The system of claim 17, wherein
each of the virtual 3D camera poses is characterized in terms of six-degrees of freedom; and
the virtual 3D camera poses are generated to cover different viewing angles with respect to the 3D model with an increment in each of the six-degrees of freedom according to a pre-determined resolution.

20. The system of claim 17, wherein the 2D features extracted from each of the virtual 2D images include one or more of:
a 2D structure corresponding to a 2D projection of the target organ in the virtual 2D image;
a mask of the 2D structure corresponding to the target organ;
a 2D ridge projected from a 3D ridge on the target organ modeled by the 3D model.

21. The system of claim 17, wherein the step of obtaining 2D feature/camera pose mapping models comprises:
pairing each of the virtual 3D camera poses with 2D features extracted from a corresponding virtual 2D image created by projecting the 3D model in accordance with a perspective determined based on the virtual 3D camera pose; and
creating the 2D feature/camera pose mapping models based on the pairs of the 2D features and the virtual 3D camera poses.

22. The system of claim 21, wherein the 2D feature/camera pose mapping models correspond to a look-up table comprising the pairs of the 2D features and the virtual 3D camera poses so that given input 2D features extracted from a 2D image, at least one 3D camera pose is identified from a pair in the look-up table that has stored 2D features similar to the input 2D features.

23. The method of claim 21, wherein the step of creating the 2D feature/camera pose mapping tools comprises:
generating training data based on the pairs of the 2D features and the virtual 3D camera poses;
performing machine learning, using the training data, to learn the 2D feature/camera pose mapping tools.

24. The system of claim 1, further comprising a camera pose estimator implemented by a processor and configured for:
receiving, during a medical procedure, a 2D image acquired by a camera inserted into a patient's body near the target object to capture surrounding information;
detecting, from the 2D image, a 2D object corresponding to the target organ and/or 2D structures corresponding to some of the 3D anatomical structures;
extracting 2D features of the detected 2D object and/or 2D structures;
predicting, based on the 2D feature/camera pose mapping models, an estimated 3D camera pose of the camera; and
projecting the 3D model to visualize the target organ and/or some of the anatomical structures associated therewith in accordance with a perspective determined based on the estimated 3D camera pose.
